(19) United States
(12) Patent Application Publication
(10) Pub. No.: US 2009/0157180 A1
(43) Pub. Date: Jun. 18, 2009

(54) MEDICAL IMPLANT CONTAINING DETECTION ENHANCING AGENT AND METHOD FOR DETECTING CONTENT LEAKAGE

(76) Inventor: Steven SCHRAGA, Surfside, FL (US)

Correspondence Address: GREENBLUM & BERNSTEIN, P.L.C., 1950 ROLAND CLARKE PLACE, RESTON, VA, 20191, US

(21) Appl. No.: 11/959,077

(22) Filed: Dec. 18, 2007

(51) Int. Cl. A61F 2/52; A61F 2/12

(52) U.S. Cl. 623/8; 623/7

(57) ABSTRACT

The present disclosure relates to implants for use in a mammalian body. The medical implant comprises a substantially impermeable outer layer enclosing a pharmaceutically acceptable content for reconstructive, plastic or cosmetic surgery. Furthermore the pharmaceutically acceptable content comprises a pharmaceutically acceptable detection enhancing agent.

### FIELD OF INVENTION

The present invention relates to the field of reconstructive, plastic and cosmetic surgery. More specifically, the invention relates to the detection of leakage, such as from ruptures or other deflation of implants used in reconstructive, plastic and cosmetic surgery.

### BACKGROUND OF THE INVENTION

Reconstructive, plastic and cosmetic surgery often utilizes medical implants for augmentation, reshaping and filling volumes in many surgical and prosthetic procedures. These procedures include medical implants comprising silicone elastomer shells which may be filled with a saline, hydrogel and/or a silicone gel.

One concern in cosmetic surgery is the potential risk of rupture of the medical implant and/or leakage of its contents into the human body. This concern has found public interest in regard to mammoplasty augmentation. Although there is a common consensus that there is no clear evidence of a causal link between the implantation of silicone breasts implants and systemic diseases, many women claim that they have become ill from their implants. Obviously, similar concerns can be expected from persons carrying implants at other locations.

The Food and Drug Administration (FDA) and the Institute of Medicine (IOM) report that breast implants may raise concerns of local complications, i.e., complications at or near the site of the implant. These complications include rupture or deflation of the implant. Reasons for rupture or deflation include normal aging of the implant, damage by surgical instruments, excessive handling during surgery, damage by other medical procedures, such as biopsy and fluid drainage, compression during mammographic imaging, stresses such as trauma or intense physical pressure, capsular contracture, overfilling or underfilling of saline-filled breast implants, or placement through an umbilical (“belly button”) incision site because it involves too much handling of the implant. Similar results are to be expected in medical implants other than breast implants but have not been studied as rigorously as breast implants in the past.

Additionally, the FDA stresses the fact that breast implants do not last a lifetime. Some implants rupture or deflate in the first few months after being implanted, some after several years and others may take ten or more years to rupture or deflate. The IOM estimates that 1-3% of saline-filled implants would rupture in the first year and this rate would increase over time, thereby having a rupture rate between 5 to 10% after ten years. Other FDA approved studies reported deflation of saline-filled breast implants at a rate of 3-5% at three years and 7-10% at five years in cases where implants were used for augmentation surgery. In cases where saline-filled implants were used for breast reconstruction, the deflation rate for saline-filled implants increased to 6-9% at three years and 8-18% at five years.

Similar studies for silicone-filled implants show a broad range of rupture rates ranging from 0.3-77%. The IOM explains this large range as being due to different types and models of implants, varying durations of implantation, different types of groups of women studied, and other factors. The IOM estimates that less than 10% of modern silicone gel-filled breast implants would have ruptured by five years and that rupture would continue to increase over time

In a study by Marotta (Marotta et al., Annals of Plastic Surgery, 49, pp. 227-247, 2002) based on numerous publications regarding instances of 9770 silicone implants, 26% of the implants were ruptured after 3.9 years, 47% were ruptures by 10.3% years and 69% were ruptured by 17.8 years.

Because silicone-gel is more viscose than saline, when a silicone gel-filled breast implant ruptures, the gel may remain contained within the fibrous capsules, i.e., the biological tissue, and often scar the tissue surrounding the implant. Thus, the rupture occurs without a visual change and the person carrying may not notice the rupture either. The effect of silicone gel in direct contact with the surrounding tissue is unknown, but there is some notion that this may cause capsular contracture. Capsular contracture happens when the scar tissue or capsule that normally forms around the implants tightens and squeezes the implant leading to an abnormal look of the breast accompanied by a tightening of the breast that may become painful to the person carrying the implant. Capsular contracture is one of the most cited reasons for removal or revision (renewed opening and/or operation at the implant site), i.e., an invasive method to detect the cause of the abnormal behavior of the surrounding tissue.

Therefore, it is desirable for a person that carries an implant as well as for the treating doctor to provide a medical implant and a method for detecting rupture and leakages as soon as the rupture occurs and most preferably before the surrounding biological tissue shows an undesired response.

### SUMMARY OF THE INVENTION

The invention makes it possible to detect ruptures or leakages from implants at the time of occurrence or shortly after they occur. With such a detection implant and method, a person with concerns regarding the implant should be able to perform a simple test (while the implant is still in the person's body) to determine if a leakage has occurred. Furthermore, since a great number reconstructive, plastic or cosmetic surgeries involve two implants, e.g., in calf, buttocks, and breast augmentations, there are generally two implants inserted. It is also desirable that the test would allow detecting which implant has the rupture and/or leakage, thereby saving unnecessary costs of removing and restoring both implants.

In one aspect, the present invention relates to a medical implant for use in a mammal body. The medical implant comprises a substantially impermeable outer layer enclosing a pharmaceutically acceptable content for reconstructive, plastic or cosmetic surgery. Furthermore the pharmaceutically acceptable content comprises a detection enhancing agent.

The detection enhancing agent may be present in a concentration sufficient to indicate a rupture in the impermeable outer layer. In embodiments, the detection enhancing agent is water-soluble and the detection enhancing agent becomes detectable upon rupture of the outer membrane and leakage of the detection enhancing agent from the medical implant.

In other embodiments of the medical implant, the detection enhancing agent may include an X-ray contrast agent, an MRI contrast agent, a fluorescent agent, a photochromic agent, a radioisotope and a dye, and/or combinations thereof.

Furthermore, in embodiments, the detection enhancing agent can be neutral in appearance, but becomes visible after leaking from a rupture of the impermeable outer layer. For example, the detection enhancing agent becomes visible by interacting with bodily fluids or by reacting with chemicals present in the body.

In most embodiments, the medical implant comprises one or more of reconstructive implants, silicone containing implants, saline containing implants, breast implants, and subcutaneous implants.

In another aspect, the present invention relates to a method for detecting a leakage from a medical implant. The method comprises implanting a medical implant containing a detection enhancing agent into a mammal body, rupturing of the medical implant, leaking of the detection enhancing agent and detecting the detection enhancing agent after rupture and/or leakage of the medical implant.

In embodiments, the leaking further may comprise leaking of the detection enhancing agent into mammal tissue; absorbing of the detection enhancing agent into a mammal circulatory system; excreting the detection enhancing agent through the renal system. The detecting may further comprise detecting a visible change in the urine. Besides detecting a visible change in the urine, the method further comprises any detecting of the detection enhancing agent in the urine.

Furthermore, in embodiments, the method may include that the leaking comprises mixing the detection enhancing agent with bodily fluids; chemical reacting of the detection enhancing agent with the bodily fluids to form a detection product. The detection product may be a dye or a photochromic or fluorescent agent. Additionally, the detection product may be water soluble.

In another aspect, the present invention relates to a method for identifying a leaking medical implant. The method comprises implanting a first medical implant containing a first detection enhancing agent and implanting a second medical implant containing a second detection enhancing agent. After rupture of any of the at least two medical implants, the faulty medical implant can be identified by detecting the first or the second detection enhancing agent.

In some embodiments, the first detection enhancing agent may be selected from a first group comprising a dye, a MRI-agent, an X-ray contrast agent, a fluorescent agent, a photochromic agent, and a radionuclide. Furthermore, the second detection enhancing agent is selected from a second group of a dye, a MRI-agent, an X-ray contrast agent, a fluorescent agent, a photochromic agent, and a radionuclide, wherein the detection enhancing agent in the first group or in the second group may be different.

In addition, the method includes that the identification of the first or the second agent may be performed by detecting at least one of the color, fluorescence, X-ray absorbance, MRI absorbance.

The present invention also relates to a method for determining and/or confirming the integrity of a medical implant. In this aspect, the method comprises implanting a medical implant containing a detection enhancing agent; monitoring the medical implant; and determining the presence or absence of a leakage.

### DETAILED DESCRIPTION OF THE INVENTION

The particulars shown herein are by way of example and for purposes of illustrative discussion of embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The present invention can be applied to any medical implant. The implant may contain any known filler. For example, the filler may be an isotonic saline solution, and/or a biocompatible silicone gel. Furthermore, the invention is also applicable to any other FDA-approved fillers including “gummy bear” implants which contain a highly viscous gel. Examples of implant fillers are well known and marketed in the U.S.A by companies such as Allergan of Santa Barbara, Calif. (formerly Inamed) and Mentor of Santa Barbara, Calif.

Medical implants are well known in the art and generally take several forms. There are single unitary medical implants which comprise a shell of physiologically inert material, such as silicone rubber or the like, which is filled with a silicone gel or a saline solution and then sealed. Inflatable mammary prostheses also are available and generally include a hollow shell of physiologically inert material, such as silicone rubber, which is implanted and then filled with a saline solution during surgery to achieve the appropriate prosthesis and breast size. In addition to the single shell inflatable mammary prosthesis, an inflatable bitumen or double shell mammary prosthesis is also available. The inflatable bitumen mammary prosthesis generally includes an inner shell filled with a gel which is positioned within an outer shell that can be filled with liquid through a valve to achieve the desired reconstructive augmentation. U.S. Pat. No. 3,293,663; U.S. Pat. No. 4,936,858 and U.S. Pat. No. 5,171,269, the entire disclosures of which are expressly incorporated by reference herein, disclose processes and methods of making implants

Suitable filler material include various materials. One example of a suitable class of materials is that which includes lipophilic triglycerides, which are disclosed in U.S. Pat. No. 6,290,723 B1, the entire disclosure of which is expressly incorporated by reference herein. Another class of materials is hydrogels, which can be formed from any hydrophilic polymeric material, for example, hydroxyalkyl acrylates. Examples of these materials can be found in U.S. Pat. No. 4,452,776, the entire disclosure of which is expressly incorporated by reference herein, describing hydrogel implant articles and methods of making the same. U.S. Pat. No. 4,279,795, the entire disclosure of which is expressly incorporated by reference herein, describes methods for polymerizing hydrogels to achieve desired properties. U.S. Pat. No. 4,056,496, the entire disclosure of which is expressly incorporated by reference herein, discloses hydrogel articles used as implants.

Another filler material can be for example, compositions made from polyoxyethylene/polyoxypropylene copolymers. This type of filler material is described in U.S. Pat. No. 5,407,445, the entire disclosure of which is expressly incorporated by reference herein.

Especially preferred articles for breast implants are silicone-filled implants as described in U.S. Pat. No. 4,573,999, the entire disclosure of which is expressly incorporated by reference herein, and U.S. Pat. No. 4,592,755, the entire disclosure of which is expressly incorporated by reference herein. Furthermore, breast prosthesis methods for making the same are disclosed in U.S. Pat. No. 5,376,117, the entire disclosure of which is expressly incorporated by reference herein, describing breast prosthesis for breast augmentations having an outer shell of a smooth non-porous material made of, for example, polyurethanes.

A medical implant for constructive, plastic or cosmetic surgery generally comprises an outer impermeable shell which is generally made of silicone elastomer and a liquid or gel as content. For decades now, silicone gel is vastly utilized.

In addition to the content of the medical implants known in the art, the present invention employs a detection enhancing agent to be incorporated into the medical implant. The detection enhancing agent is chosen to become detectable when rupture of leakage of the medical implant occurs. Any pharmaceutical detection enhancing agent is applicable. For example, the detection enhancing agent may comprise X-ray contrast agents, MRI contrast agents, fluorescent agents or radioisotopes. Furthermore, the scope of this invention also includes detection enhancing agents that have properties such that the person who carries the medical implant may visually detect the leakage or rupture. Such detection enhancing agents comprise photochromic agents, i.e., chemical compounds that change color upon irradiation with light; dyes or leuco derivatives of dyes, and pH-indicators.

Leuco derivatives of dyes are colorless or almost colorless compounds that can chemically rearrange or chemically react to form a dye. For example (and without limiting the invention), ninhdrin is initially a colorless substance. Upon exposure to amino groups as they are present in biomolecules, e.g., in the amino acid lysine, ninhydrin reacts to form a Schiff base. This Schiff base generally has a distinct blue color when sufficient amino groups are present for the reaction to occur. The reaction can be displayed by the following sequence:

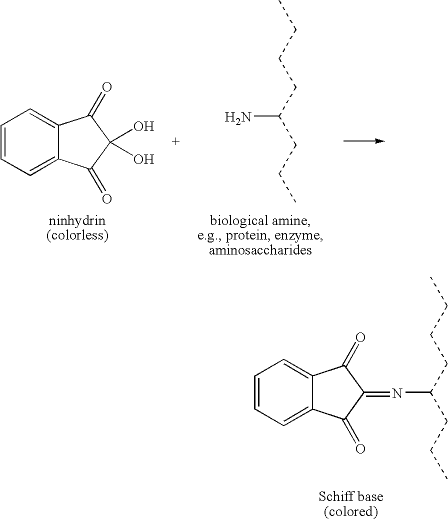

In some embodiments, the visual detection can become eminent upon rupture or leakage of the contents of a medical implant. Therefore, the detection enhancing agents should be colorless or almost colorless when distributed throughout a medical implant in order to not interfere with the cosmetic function or appearance of the site of implantation. Upon rupture or leakage of the implant, the detection enhancing agent should become visible, for example, as described above for ninhydrin with free amino groups. Other ways of becoming visible include change of a pH as the detection enhancing agent leaks from the implant into the mammal body that triggers a pH indicator to change color. One example of this type of pH-indicator, but not limiting the scope of the invention thereto, is phenolphthalein, which is colorless in acidic environments, but becomes purple in alkaline environments.

Another class of detection enhancing agents are ordinary dyes that are insoluble in the gel that forms the content of the implant, thereby forming an suspension with the gel. This suspension may be colorless or appear almost colorless, i.e., it does not change the appearance of the implant to such an extent that it would interfere with the implant's cosmetic function. Upon rupture or leakage of the implant, the suspension comes in contact with other liquids such as water, and the dyes dissolve and show their full color in solution.

Another alternative of type of dye that forms a suspension in the implant and becomes visible upon rupture or leakage and salvation into fluids is a two component dye. The two component dye comprises different components which are colorless or almost colorless and become visible upon mixing in solution. One example of these components is a transition metal complex that includes a central metal ion and a ligand. The metal ion can be provided as plain inorganic salt, e.g., a chloride, bromide, sulfate, or phosphate or organic salt, e.g., an acetate, propionate, butanoate, caprylate, etc. The ligand with which the metal ion forms a colored complex can be provided either as a salt or unmodified. One example, without limiting the scope of the invention thereto is a medical implant that contains a suspension of copper acetate and also contains chlorophyll. Upon rupture and leakage of the medical implant, copper acetate and chlorophyll would dissolve in bodily fluids and form a colored complex, which would become visible.

Another type of compounds that would indicate rupture and leakage of a medical implant are photochromic agents. Photochromic compounds are molecules that are present in two forms. One form is a colorless or almost colorless leuco form, the second form is the colored form. Generally the leuco form rearranges into the colored form upon UV excitation. By definition, this rearrangement is reversible, i.e., the colored form reverts back to the leuco form. A general photochromic reaction can be illustrated:

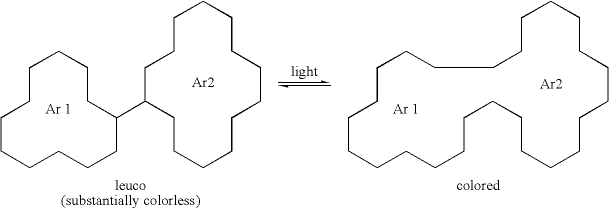

Generally, two aromatic systems, Ar1 and Ar2, are synthesized to form two independent π-electron rich moieties in the same molecule. These two moieties are usually connected either via a single bond (σ-bond) or via one central carbon atom (spiro-form). The system with two independent aromatic moieties, here depicted as leuco-form is colorless or substantially colorless, i.e., the presence of the leuco-form itself does not undesirably alter the color of its environment like the content of the implant. Upon irradiation with light or UV-light, the leuco-from rearranges to from a single π-electron system, which is colored. Regions Ar1 and Ar2 are now connected through π-electrons. The color of this single π-electron system can be tuned depending on the number of π-electrons in the system and the type of substitutions on the regions Ar1 and Ar2. Upon cease of irradiation, the colored form reverts back to the leuco-form, thereby becoming colorless or substantially colorless.

Photochromic systems have been well studied. For example, U.S. Pat. No. 6,891,038 B2, which is expressly incorporated by reference herein in its entirety, discloses spiro(indoline) naphthoxazines, which are compounds whose leuco-form includes a spiro carbon as displayed by compounds of formula (I). Also shown are the regions of the leuco-form pertaining to Ar1 and Ar2 in the general reaction equation above:

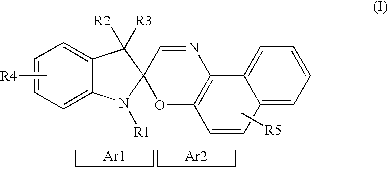

Substituents R1 through R5 can be any chemical group. These substituents can be chosen to tune the enthalpy level of the leuco-form, i.e., determining what wavelength of light is required to render the leuco-form into the colored form. Some species can be chosen to react at general visible light, other species can be chosen to react at U.V. light only. Furthermore, subsituents R1 through R5 can also have a linker function, wherein the photochromic compound is chemically bond into the polymeric material of the implant contents. Another function of the substituents lies in the specific color of the colored form. Here, the subsituents can be chosen to display a colored form that distinguishes from any color of tissue and biological fluids of the person carrying the implant. Of course, although function and type of the substituents are discussed on the example of (spiro)indoline napthoxazine compounds of formula (I), same functions and type of any substituents of other photochromic compounds are contemplated.

U.S. Pat. No. 3,973,966, the entire disclosure of which is expressly incorporated by reference, discusses photochromic diphenyl dibenzochromenes as displayed in formula (II):

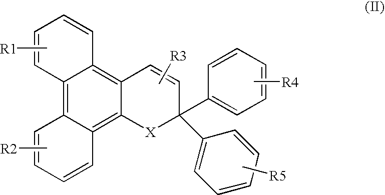

Here, X can be O, S, Se, or NH. As described above, here again substituents R1 through R5 can be chosen to tune the photochromic properties, link the compound with a polymer, or modify the compound in any other way to render the compound suitable as a detection enhancing agent, e.g., tune the solubility of the compound as desired for the purpose. For example, if a more water soluble compound is desired, then the R1 through R5 can independently chosen to include water-solubilizing groups.

Applications for photochromic compounds are multifaceted including application of these compounds in biological tissue. For example, U.S. Pat. No. 7,169,941 B2 and U.S. Pat. No. 4,979,976, the entire disclosures of which are expressly incorporated by reference herein, describe optical lenses and glasses containing photochromic compounds. In U.S. Pat. No. 5,698,020, the entire disclosure of which is expressly incorporated by reference, photochromic compounds in dental materials are disclosed, exemplifying the cosmetic use of these compounds in humans. Another example for the use of photochromic compounds in humans is described in U.S. Pat. No. 6,470,891 B2, the entire disclosure of which is expressly incorporated by reference herein, describing photochromic tattoos.

With respect to combining these materials with a polymeric network, U.S. Pat. No. 7,041,763 B2, the entire disclosure of which is expressly incorporated by reference herein, describes preparation and methods for preparing photochromic polymers.

Upon rupture and leakage of the medical implant, the detection enhancing agent will enter the mammalian system. It is within the scope of the invention that any detection enhancing agent can be absorbed into the cardiovascular system and excreted through the renal system. The following discussion exemplifies the use of photochromic agents. Upon excretion of the photochromic detection enhancing agent either through the skin or through the renal system, a sample of the skin or of the urine can be investigated such as by irradiation with light to detect the colored form of the photochromic agent. Any conventional irradiation is possible to check the presence of the photochromic detection enhancing agent on the skin or in the urine. For example, exposing the sample to a light bulb or a UV-lamp or to sunlight will convert the leuco form into the colored form.

Typically, medical implants of the invention comprise an impermeable outer layer or shell. In some embodiments, the outer layer may comprise any conventional polymer known in the medical discipline for usage as an implant outer layer. For example, the outer layer can comprise a silicone elastomer. This impermeable outer layer is preferably impermeable as to the contents of the medical implant as well as fluids and liquids inside and outside of the medical implant. The medical implant may further comprise a pharmaceutical content. Any conventional content known in the art may be used. For example and without limiting the inventions thereto, the content may comprise a salt water solution or saline, or a silicone gel. Of course, it is imperative that the outer layer and the content are medical and pharmaceutically acceptable, i.e., the materials are chosen that they do not cause an immune response in the body. Therefore, the materials are to be sterile and modified in such way that the body does not react adversely, a phenomenon named capsular contracture. In addition to these elements, the medical implant further comprises the pharmaceutically acceptable detection enhancing agent.

The pharmaceutically acceptable detection enhancing agent should be present at a concentration or amount sufficient to indicate a rupture in the impermeable outer layer. The sufficient amount or concentration depends on the type of the detection enhancing agent used and the size of the medical implant. For example, if the detection enhancing agent is a contrast agent for X-ray or MRI monitoring the sufficient concentrations are different than when a photochromic agent or a dye is used. However, depending on the type of the detection enhancing agent and the size of the medical implants, sufficient concentrations and amounts can be determined by the practitioner in the field.

Furthermore, in embodiments, the detection enhancing agent may be water soluble and or soluble in biological fluids. The advantage of such a property is that the detection enhancing agent is removed naturally after leaving the medical implant, either through the cardiovascular and renal system or through perspiration. Another advantage is that the person carrying a rupture implant may be able to detect the detection enhancing agent by visually examining the skin at the location of the implant or by examining the urine.

The detection enhancing agent can be any known pharmaceutical detection enhancing agent. In embodiments, the detection enhancing agent may comprise an X-ray contrast agent, an MRI contrast agent, a fluorescent agent, a photochromic agent, a radioisotope or a dye.

Preferably, the detection enhancing agent is neutral in appearance, but becomes visible after leaking from the rupture of the impermeable outer layer. In some embodiments, the detection enhancing agent may be a photochromic agent that is present in its leuco form inside the implant and the mammal body. Upon exiting the body and exposure to light the photochromic agent turns into its colored form and thus detectable by the person carrying the implant. Another example of how the detection enhancing agent may become visible may include the use of a pH-indicator, wherein the indicator inside the medical implant is colorless, but becomes colored when leaking through the rupture based on a different pH-value of the media outside of the implant.

Still another embodiment of the present invention for a detection enhancing agent to become visible can result from a dye which is insoluble inside the medical implant, e.g., it is insoluble in saline or silicone gel but is soluble in fluids and liquids of the body. In this embodiment, the dye may be distributed as a fine particulate throughout the medical implant, thereby resembling a suspension or a sol, which would display no color or only a faint color of the dye. Upon rupture of the shell and leakage of the dye through the outer wall, the dye would dissolve in bodily fluids and display a detectable color.

Yet another embodiment of the present invention comprises a method for determining the implant integrity by monitoring a medical implant and determining the presence or absence of a leakage or rupture. The detection enhancing agents that are applicable to this method are detectable by use of auxiliary equipment. For example, if the detection enhancing agent is an X-ray contrast agent or an MRI contrast agent, leakage of the detection enhancing agent from the rupture can be localized with an MRI scanner or by X-ray medical imaging. Furthermore, the absence of a leakage or rupture can also be determined, for example, by evaluating the size, shape, concentration, or density displayed by the detection enhancing agent after medical imaging or scanning. An intact medical implant would show a scan or medical image consistent with the size or shape having an undamaged edge, wherein the detection enhancing agent is homogenously distributed throughout the volume of the medical implant. Any known X-ray contrast agent can be used. For example, Barium compounds find applications as such contrast agents. Furthermore, any known MRI agent can be used. For example, gadolinium or iron compounds are known to be MRI image enhancers.

MRI agents that can be used with the implant include paramagnetic contrast agents, e.g., ferric chloride, ferric ammonium citrate, and other ferric complexes, for example ferric porphyrines; gadolinium complexes, for example, gadolinium-DTPA; manganese salts and complexes. The MRI agents may be chemically, i.e., covalently attached to the filler or be present in mixture therewith.

In embodiments, medical imaging by either X-ray or MRI of a medical implant containing one of the two discussed contrast agent, respectively, will reveal if rupture and leakage of the contents of medical implant occurred. Furthermore, these methods will also give more accurate information regarding the exact location of a rupture, the rate of diffusion of the contents through the leak, the tissue absorbing the content of the implant and the scopes of organs affected by the leak.

As yet further examples for detection enhancing agents that are within the scope of the present invention are fluorescent compounds and compounds containing a radioisotope. The advantage of these compounds is that for the most part, they do not have a color of their own, which make them useful in reconstructive and plastic surgery involving implants that are inserted subcutaneously with little tissue to cover the implant. Rupture and leakage of the implant may then be detected through analysis of the implant location (direct examination) or through analysis of the urine. Fluorescent compounds can be detected by the use of an UV lamp. Similarly, a compound containing a radioisotope can be detected by any known medical method, for example, by emission tomography or X-ray imaging. Any known pharmaceutical acceptable fluorescent agent or radionuclide can be used.

The use of a radioisotope may be appropriate for applications in specific circumstances. For example it is within the scope of the invention that upon the notion or certitude that a medical implant is ruptured, a medical practitioner may inject a radiopharmaceutical or radioisotope into the medical implant to monitor the course of leakage of the contents through the body in order to evaluate measures to remedy the damages.

The present invention also includes a method for detecting a leakage from a medical implant. A medical implant containing a detection enhancing agent is implanted into the body of a mammal. The methods include any known medical implant insertion method. For example, the implanting can be done subcutaneously, subglandularly, subfascially, submuscularly. subpectorally, or any other conventional placement of the implant.

Rupture of the medical implant can occur by any cause. For example, rupture can occur during implantation of the medical implant or medical procedures near the location of the implant, by trauma or pressure onto the area of the medical implant, or by material design and age.

Leaking of the detection enhancing agent may include continuous and sporadic leaking. It is also contemplated within the scope of the invention that leaking may not originate from one single rupture but also may occur from various ruptures, for example, as they may occur when the medical implant is subject to extraordinary pressures, shear and/or tear forces.

Detecting of the leaking may be conducted as described above for the various detection enhancing agents. The detecting may comprise visually detecting by the person carrying the medical implant and also detection by a medical practitioner with the use of auxiliary equipment such as an MRI scanner, an X-ray imaging or detection device, or a fluorescence spectrometer.

The method further comprises the fact that the detection enhancing agent may be absorbed by the tissue surrounding the medical implant and transported into the cardiovascular system for excretion through the kidneys. Therefore, the detection enhancing agent or desired metabolites thereof can be identified in the urine. For example, contrast agents used in MRI imaging are well known and injected into various vascular systems or organs of the body. Therefore, the use of such agents as a detection enhancing agent in a medical implant is contemplated as well. Furthermore, the present method also includes that the detection enhancing agent becomes visible upon leakage, i.e., a leuco agent turns into a colored agent or a precursor of dye is metabolized into the dye. Furthermore, it is also contemplated within the scope of this invention that the detection enhancing agent reacts with compounds present in the body to form a detection product. For example, a detection enhancing agent may comprise a protein dye or protein marker, which reacts with proteins in the bodily fluids to form a detection product that becomes visible and detectable upon excretion.

It is desired that such a detection product be water soluble in order to facilitate fast excretion through the kidneys and accelerated detection. Besides a protein marker, markers of other biomolecules are considered as well. Furthermore, the group of markers is not only within the scope of dyes, but also other compounds such as, for example, fluorescent markers.

The present invention does not relate to only detection of a leak in a medical implant. Many implants are inserted in duplicate for symmetry reasons. Furthermore, in extensive reconstruction surgery, there may be more than only two implants being used to achieve the reconstructive result. If all implants would carry the same detection enhancing agent, that would be detected indirectly, e.g., through the renal system, the person carrying the medical implant or the practitioner analyzing a urine sample or other fluid sample could not determine which implant has the actual leak.

Therefore, this invention also relates to methods for identifying a leak of one medical implant in a body carrying several implants. This method comprises implanting at least two medical implants wherein each of the medical implants carries different detection enhancing agents.

The detection enhancing agents thus may also differ in type. For example, one agent in the first implant can be a fluorescent agent, the second implant can have a protein dye marker as a detection enhancing agent. Alternatively, the detection enhancing agent may be of the same type but have differentiating read-outs. For example, two medical implants can both carry photochromic agents as a detection enhancing agent. However the first implant has a photochromic agent that gives a red color as a response, while the second gives a blue color as response. Identifying either one of the two colors will determine which implant is leaking. Identifying a mixture of the two colors, e.g., purple, will identify leakage in both implants.

The exact identification of a leaking implant will help the medical surgeon to decide which implant to replace, or in the alternative, if several implants are leaking, suggest which one to replace first based on the amount of detection enhancing agents identified from the leakage.

What is claimed is:

1. An implant for use in a mammalian body, comprising:
an outer layer enclosing a pharmaceutically-acceptable content for reconstructive, plastic or cosmetic surgery; and a detection enhancing agent.

2. The implant according to claim 1, wherein the detection enhancing agent is present in an amount sufficient to indicate a rupture in the impermeable outer layer.

3. The implant according to claim 2, wherein the detection enhancing agent is water-soluble and the detection enhancing agent is detectable upon rupture of the outer membrane and leakage of the detection enhancing agent from the medical implant.

4. The implant according to claim 1, wherein the detection enhancing agent is at least one of an X-ray contrast agent, an MRI contrast agent, a fluorescent agent, a photochromic agent, a radioisotope and a dye.

5. The implant according to claim 1, wherein the detection enhancing agent is neutral in appearance, but becomes detectable after leaking from a rupture of the impermeable outer layer.

6. The implant according to claim 5, wherein the detection enhancing agent becomes detectable by at least one of interacting with bodily fluids and by reacting with chemicals present in the body.

7. The implant according to claim 1, wherein the medical implant comprises at least one of reconstructive implants, silicone containing implants, saline containing implants, breast implants, and subcutaneous implants.

8. A method for detecting leakage from an implant comprising:
implanting into a mammalian body a medical implant containing a detection enhancing agent;
leaking of the detection enhancing agent; and
detecting the detection enhancing agent.

9. The method according to claim 8, wherein the leaking further comprises
leaking of the detection enhancing agent into mammalian tissue;
absorbing of the detection enhancing agent into a mammal circulatory system;
excreting the detection enhancing agent through the renal system.

10. The method according to claim 9, wherein the detecting further comprises detecting a visible change in urine.

11. The method according to claim 9, wherein the detecting further comprises detecting the detection enhancing agent in urine.

12. The method according to claim according to claim 8, wherein the leaking further comprises
mixing the detection enhancing agent with bodily fluids;
a chemical reaction of the detection enhancing agent with the bodily fluids to form a detection product.

13. The method according to claim 12, wherein the detection product is a dye.

14. The method according to claim 12, wherein the detection product is a photochromic or fluorescent agent.

15. The method according to claim 12, wherein the detection product is water soluble.

16. A method for identifying a leaking medical implant comprising:
implanting a first medical implant containing a first detection enhancing agent;
implanting a second medical implant containing a second detection enhancing agent;
identifying the first or the second detection enhancing agent after leakage from the first or second medical implant.

17. The method according to claim 16, wherein the first detection enhancing agent is selected from a first group of a dye, a MRI-agent, an X-ray contrast agent, a fluorescent agent, a photochromic agent, and a radionuclide.

18. The method according to claim 17, wherein the second detection enhancing agent is selected from a second group of a dye, a MRI-agent, an X-ray contrast agent, a fluorescent agent, a photochromic agent, and a radionuclide.

19. The method according to claim 16, wherein the identifying of the first or the second agent is performed by detecting color, fluorescence, X-ray absorbance, MRI absorbance.

20. A method for determining an implant integrity of medical implants, comprising:
implanting a medical implant containing a detection enhancing agent;
monitoring the medical implant; and
determining the presence or absence of a leakage.
